(19) United States
(12) Patent Application Publication
(10) Pub. No.: US 2026/0327875 A1
(43) Pub. Date: Oct. 22, 2026

(54) Method for Forming Multilayer Nonwoven Materials with Topographic Projections Via Differential Shrinkage

(71) Applicant: Kimberly-Clark Worldwide, Inc., Neenah, WI (US)

(72) Inventors: Stephen A. Marrano, Oshkosh, WI (US); Jian Qin, Appleton, WI (US); Frank P. Abuto, Johns Creek, GA (US); Stephen M. Lindsay, Appleton, WI (US); Sara L. Rosack, Greenville, WI (US); Sridhar Ranganathan, Alpharetta, GA (US); Daniel T. Labash, Appleton, WI (US); Greg J. DeGrave, Combined Locks, WI (US); Andrew E. Neubauer, Fremont, WI (US)

(21) Appl. No.: 19/099,579

(22) PCT Filed: Jul. 28, 2023
(86) PCT No.: PCT/US2023/028968

§ 371 Date: Jan. 29, 2025

Related U.S. Application Data
(60) Provisional application No. 63/393,547, filed on Jul. 29, 2022.

(51) Int. Cl. D04H 1/72; D04H 1/26; D04H 1/407; D04H 1/435; D04H 1/4374; D04H 1/541; D04H 1/55; D04H 1/559

(52) U.S. Cl. CPC D04H 1/72; D04H 1/26; D04H 1/407; D04H 1/435; D04H 1/4374; D04H 1/5418; D04H 1/55; D04H 1/559; D10B 2331/041; D10B 2403/0111; D10B 2509/026

(57) ABSTRACT

A process and system for producing nonwoven materials are disclosed. A foam formed nonwoven web includes a first layer and a second layer. The first layer is shrunken relative to the second layer such that the first layer forms a plurality of projections at an outer surface of the first layer that faces away from the second layer.

### BACKGROUND

Many different types of nonwoven materials exist that are designed to have different functions. In many embodiments, the nonwoven materials are designed to have liquid handling properties. These nonwoven materials can be used in absorbent articles or used to produce wiping products, tissue products, and the like. Absorbent articles, also referred to as personal care products, such as diapers, diaper pants, training pants, adult incontinence products, and feminine care products, can include a variety of substrates. For example, a diaper can include an absorbent structure, nonwoven materials, and films. Similarly, facial tissues, wipes, and wipers can also include various substrates.

In certain applications, multilayer nonwoven materials include an acquisition layer for receiving fluids. In general, the acquisition layer preferably has a high void volume and compression resistance. However, conventional methods for forming nonwoven materials offer limited void volume and compression resistance in the acquisition layer. Improved methods for forming nonwoven materials with an acquisition layer having a high void volume and compression resistance would be useful.

### SUMMARY

In general, the present disclosure is directed to a nonwoven material and process for creating the nonwoven material. In a multilayer, foam formed nonwoven web, such as a bilayer nonwoven web, adjacent layers may be at least partially structurally interconnected (e.g., fiber entanglements between the adjacent layers, gradient interfaces between the layers, interlayer adhesion, etc.), and the the nonwoven web may be heated such that one of the layers contracts to a lesser extent than at least one other layer, which can cause the one layer to deform and produce a plurality of projections. As an example, a first layer of the nonwoven web may be at least partially (e.g., entirely) cured and/or dried while the first layer is under tension. The first layer may correspond to an acquisition layer of the nonwoven web. Curing the first layer may define a structural relationship between the first layer and a second, different layer of the nonwoven web. For instance, at least partially curing the first layer may be define the interlayer adhesion between the first and second layers. After the top layer is at least partially cured, the second layer may be cured and/or dried while the second layer is under tension. The tension in the first layer and/or the second layer may be controlled during the curing such that shrinkage of the second layer deforms the first layer such that topographical projections are formed on an outer surface of the first layer. As will be described in greater detail below, example aspects of the present disclosure offer many different advantages and benefits. For instance, an acquisition layer of the nonwoven material may advantageously have a high void volume and compression resistance due to the topographical projections.

In example aspects, various mechanisms may be utilized to form the topographical projections via differential shrinkage between the first and second layers. For instance, the second formed layer may include a fiber capable of hydrogen bonding. Shrinkage of the second layer may be due at least in part to capillary condensation and hydrogen bonding during drying. As another example, the second formed layer may include superabsorbent particles that partially swell during processing in the wet end of the foam forming process. Shrinkage of the second layer during drying may be due at least in part to shrinkage of the superabsorbent particles as water is removed. As another example, the second layer may include a fiber or other material that shrinks upon heating, such as PLA staple fibers. Shrinkage of the second layer during heating may be due at least in part to shrinkage of the heat-shrinkable material. Various combinations of hydrogen-bond-capable fibers, superabsorbent, and/or heat-shrinkable fiber or other material may also be utilized. Thus, e.g., shrinkage of the second layer may be due to a combination of these mechanisms.

In one example embodiment, a method for forming a nonwoven material includes: foam forming a nonwoven web with a plurality of layers; at least partially curing a first layer of the plurality of layers while the nonwoven web is in tension; after at least partially curing the first layer, curing a second layer of the plurality of layers while the nonwoven web is in tension; and relaxing the nonwoven web such that the second layer contracts and the first layer deforms to produce a plurality of projections at an outer surface of the first layer that faces away from the second layer.

In another example embodiment, a nonwoven material includes a foam formed nonwoven web with a first layer and a second layer. The first layer is shrunken relative to the second layer such that the first layer forms a plurality of projections at an outer surface of the first layer that faces away from the second layer.

In another example embodiment, a method for forming a nonwoven material includes: foam forming a nonwoven web with a plurality of layers; heating a first layer of the plurality of layers in the nonwoven web while the nonwoven web is in tension, a water content in the first layer decreasing during the heating of the first layer; after heating the first layer and while the nonwoven web is in tension, heating a second layer of the plurality of layers in the nonwoven web, a water content in the second layer decreasing during the heating of the second layer; and, after heating the second layer, relaxing the nonwoven web such that the first layer deforms with a plurality of projections at an outer surface of the first layer that faces away from the second layer.

In another example embodiment, a method for forming a nonwoven material includes: foam forming a nonwoven web with a plurality of layers, a first layer and a second layer of the plurality of layers at least partially interconnected; and heating the nonwoven web such that the second layer contracts less than the first layer, the first layer deforming in order to produce a plurality of projections at an outer surface of the first layer that faces away from the second layer.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 is a schematic view of a system for producing nonwoven materials according to an example embodiment of the present disclosure;

FIG. 2 is a schematic view of a component feed system of the example system of FIG. 1;

FIG. 3 illustrates a method for forming a nonwoven material according to an example embodiment of the present subject matter.

FIG. 4 illustrates a method for forming a nonwoven material according to another example embodiment of the present subject matter.

FIG. 5 is a side, elevation view of a nonwoven material according to an example embodiment of the present subject matter, with the nonwoven material under tension.

FIG. 6 is a side, elevation view of the example nonwoven material of FIG. 5, with the nonwoven material in a relaxed state.

FIG. 7 is a perspective view of a nonwoven material according to an example embodiment of the present subject matter.

FIG. 8 is a partial side, elevation view of the example nonwoven material of FIG. 7.

FIG. 9 is a perspective view of a nonwoven material according to an example embodiment of the present subject matter.

Repeat use of reference characters in the present specification and drawings is intended to represent the same or analogous features or elements of the disclosure.

### DETAILED DESCRIPTION

It is to be understood by one of ordinary skill in the art that the present discussion is a description of exemplary embodiments only and is not intended as limiting the broader aspects of the present disclosure.

Each example is provided by way of explanation and is not meant as a limitation. For example, features illustrated or described as part of one embodiment or figure can be used on another embodiment or figure to yield yet another embodiment. It is intended that the present disclosure include such modifications and variations.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. As used herein, the terminology of “first,” “second,” “third”, etc. does not designate a specified order, but is used as a means to differentiate between different occurrences when referring to various features in the present disclosure. Many modifications and variations of the present disclosure can be made without departing from the spirit and scope thereof. Therefore, the exemplary embodiments described herein should not be used to limit the scope of the invention.

As used herein, the term “foam formed product” means a product formed from a suspension including a mixture of a solid, a liquid, and dispersed gas bubbles.

As used herein, the term “foam forming process” means a process for manufacturing a product involving a suspension including a mixture of a solid, a liquid, and dispersed gas bubbles.

As used herein, the term “foaming fluid” means any one or more known fluids compatible with the other components in the foam forming process. Suitable foaming fluids include, but are not limited to, water.

As used herein, the term “foam half life” means the time elapsed until the half of the initial frothed foam mass reverts to liquid water.

As used herein, the term “layer” refers to a structure that provides an area of a material or substrate in a height direction of the substrate that is comprised of similar components and structure.

As used herein, the term “nonwoven web” means a web having a structure of individual fibers or threads which are interlaid, but not in an identifiable manner as in a knitted web.

As used herein, unless expressly indicated otherwise, when used in relation to material compositions the terms “percent”, “%”, “weight percent”, or “percent by weight” each refer to the quantity by weight of a component as a percentage of the total except as whether expressly noted otherwise.

The term “personal care absorbent article” refers herein to an article intended and/or adapted to be placed against or in proximity to the body (i.e., contiguous with the body) of the wearer to absorb and contain various liquid, solid, and semi-solid exudates discharged from the body. Examples include, but are not limited to, diapers, diaper pants, training pants, youth pants, swim pants, feminine hygiene products, including, but not limited to, menstrual pads or pants, incontinence products, medical garments, surgical pads and bandages, and so forth.

The term “superabsorbent material” as used herein refers to water-swellable, water-insoluble organic or inorganic materials including superabsorbent polymers and superabsorbent polymer compositions capable, under the most favorable conditions, of absorbing at least about 10 times their weight, or at least about 15 times their weight, or at least about 25 times their weight in an aqueous solution containing 0.9 weight percent sodium chloride.

The term “machine direction” as used herein refers to the direction of travel of the forming surface onto which fibers are deposited during formation of a nonwoven web.

The term “cross-machine direction” as used herein refers to the direction which is perpendicular to both the machine direction and the height direction, e.g., that is perpendicular to the machine direction and the cross-machine direction.

The term “pulp” as used herein refers to fibers from natural sources such as woody and non-woody plants. Woody plants include, for example, deciduous and coniferous trees. Non-woody plants include, for example, cotton, flax, esparto grass, milkweed, straw, jute, hemp, and bagasse. Pulp fibers can include hardwood fibers, softwood fibers, and mixtures thereof.

The term “average fiber length” as used herein refers to an average length of fibers, fiber bundles and/or fiber-like materials determined by measurement utilizing microscopic techniques. A sample of at least 20 randomly selected fibers is separated from a liquid suspension of fibers. The fibers are set up on a microscope slide prepared to suspend the fibers in water. A tinting dye is added to the suspended fibers to color cellulose-containing fibers so they may be distinguished or separated from synthetic fibers. The slide is placed under a Fisher Stereomaster II Microscope—S19642/S19643 Series. Measurements of 20 fibers in the sample are made at 20× linear magnification utilizing a 0-20 mils scale and an average length, minimum and maximum length, and a deviation or coefficient of variation are calculated. In some cases, the average fiber length will be calculated as a weighted average length of fibers (e.g., fibers, fiber bundles, fiber-like materials) determined by equipment such as, for example, a Kajaani fiber analyzer Model No. FS-200, available from Kajaani Oy Electronics, Kajaani, Finland. According to a standard test procedure, a sample is treated with a macerating liquid to ensure that no fiber bundles or shives are present. Each sample is disintegrated into hot water and diluted to an approximately 0.001% suspension. Individual test samples are drawn in approximately 50 to 100 ml portions from the dilute suspension when tested using the standard Kajaani fiber analysis test procedure. The weighted average fiber length may be an arithmetic average, a length weighted average or a weight weighted average and may be expressed by the following equation:

$${\sum }_{{x}_{i} = 0}^{k} ( {x}_{i} * {n}_{i} ) / n$$

where
k=maximum fiber length
xᵢ=fiber length
nᵢ=number of fibers having length xi
n=total number of fibers measured.

One characteristic of the average fiber length data measured by the Kajaani fiber analyzer is that it does not discriminate between different types of fibers. Thus, the average length represents an average based on lengths of all different types, if any, of fibers in the sample.

As used herein the term “staple fibers” means discontinuous fibers made from synthetic polymers or regenerated cellulose, such as polypropylene, polyester, post consumer recycle (PCR) fibers, polyester, nylon, viscose, rayon, and the like, and those not hydrophilic may be treated to be hydrophilic. Staple fibers may be cut fibers or the like. Staple fibers can have cross-sections that are round, bicomponent, multicomponent, shaped, hollow, or the like.

In general, the present disclosure is directed to a process and system for producing nonwoven materials and to a nonwoven material. Moreover, the present disclosure is directed to methods and systems that can produce nonwoven materials through foam-forming. The nonwoven material may be a multi-layer material, such as a bilayer nonwoven material. In accordance with example aspects of the present disclosure, the nonwoven materials may be formed with three-dimensional topography on an outer surface of one of the layers of the nonwoven materials. The three-dimensional topography may provide many advantages and benefits. The nonwoven materials of the present disclosure may be used in numerous and diverse end use applications. The three-dimensional topography may provide significant improved functionality that is tailored for the particular application.

In one example aspect, for instance, the three-dimensional topography may form bumps that advantageously increase void volume and increase compression resistance of an acquisition layer in absorbent articles, wiping products, or the like. The three-dimensional topography may also form fluid control canals that can be used to quickly intake and distribute fluids, which can provide advantages when incorporated into absorbent articles, wiping products and the like.

In addition to the above, the three-dimensional topography may significantly improve the breathability of the material. Nonwoven materials made according to example aspects of the present disclosure may produce an optimum microclimate when contained in an absorbent article by allowing better air exchange at an interface between the three-dimensional topography and the skin of a wearer. When incorporated into a diaper, an adult incontinence product, or the like, the three-dimensional topography may be configured to collect and move away from the body feces and other bodily fluids for protecting the skin and providing a garment that promotes better health. The three-dimensional topography may also contribute to a drier skin due to reduced contact area between the three-dimensional topography and the skin of a wearer.

Nonwoven materials made according to the present disclosure can also provide enhanced comfort. For instance, the three-dimensional topography may provide a “cushion-like” feel to users. The three-dimensional topography may also be configured for visual appeal or as a signal to a user to highlight some other benefit of the product.

Nonwoven materials made according to example aspects of the present disclosure may be formed of fibers and optionally other solid material via a foam forming process. The nonwoven materials may be a multi-layer material, such as a bilayer material.

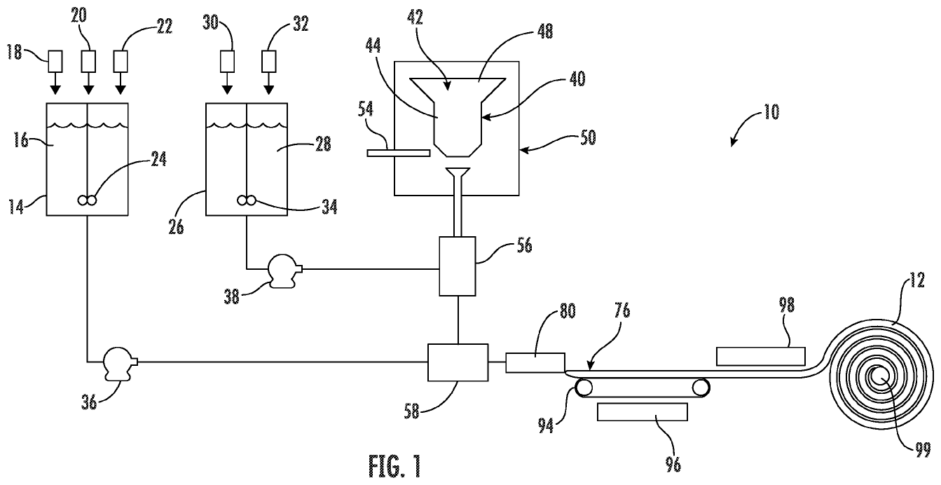

Referring to FIG. 1, for exemplary purposes only, one embodiment of a process that may be used to form nonwoven materials or substrates in accordance with example aspects of the present disclosure is shown. The process and system as illustrated in FIG. 1 can be a foam forming process. As shown in FIG. 1, an apparatus 10 is configured for forming a substrate 12. Moreover, apparatus 10 may be configured as part of a foam forming process to manufacture a substrate 12 that is a foam formed product. The apparatus 10 may include a first tank 14 configured for holding a first fluid supply 16. In some example embodiments, the first fluid supply 16 may be a foam. The first fluid supply 16 may include a fluid provided by a supply of fluid 18. In some example embodiments, the first fluid supply 16 may include a plurality of fibers provided by a supply of fibers 20; however, in other example embodiments, the first fluid supply 16 may be free from or substantially free from a plurality of fibers. The first fluid supply 16 may also include a surfactant provided by a supply of surfactant 22. In some example embodiments, the first tank 14 may include a mixer 24, as will be discussed in more detail below. The mixer 24 is operable to mix (e.g., agitate) the first fluid supply 16 in order to mix the fluid, fibers (if present), and surfactant with air, or some other gas, to create a foam. The mixer 24 may also mix the foam with fibers (if present) to create a foam suspension of fibers in which the foam holds and separates the fibers to facilitate a distribution of the fibers within the foam (e.g., as an artifact of the mixing process in the first tank 14). Uniform fiber distribution can promote desirable substrate properties including, for example, strength and the visual appearance of quality.

In certain example embodiments, the apparatus 10 may also include a second tank 26 configured for holding a second fluid supply 28. In some example embodiments, the second fluid supply 28 may be a foam. The second fluid supply 28 may include a fluid provided by a supply of fluid 30 and a surfactant provided by a supply of surfactant 32. In some example embodiments, the second fluid supply 28 may include a plurality of fibers in addition to or as an alternative to the fibers present in the first fluid supply 16. In some example embodiments, the second tank 26 may include a mixer 34. The mixer 34 may be operable to mix the second fluid supply 28 in order to mix the fluid and surfactant with air, or some other gas, to create a foam.

For either or both the first tank 14 and the second tank 26, the first fluid supply 16 or the second fluid supply 28 may be operated to form a foam. In some example embodiments, the foaming fluid and other components may be acted upon so as to form a porous foam having an air content greater than about fifty percent (50%) by volume and desirably an air content greater than about sixty percent (60%) by volume. In certain example aspects, a highly expanded foam is formed having an air content of between about sixty percent (60%) and about ninety-five percent (95%) by volume and in further example aspects between about sixty-five percent (65%) and about eighty-five percent (85%) by volume. In certain example embodiments, the foam may be acted upon to introduce air bubbles such that a ratio of expansion (volume of air to other components in the expanded stable foam) is greater than 1:1 and in certain embodiments the ratio of air to other components may be between about 1.1:1 and about 20:1 or between about 1.2:1 and about 15:1 or between about 1.5:1 and about 10:1 or even between about 2:1 and about 5:1.

The foam may be generated by one or more mechanisms and methods known in the art. Examples of suitable methods include, without limitation, aggressive mechanical agitation, such as by mixers 24, 34, injection of compressed air, and so forth. Mixing the components through the use of a high-shear, high-speed mixer is particularly well suited for use in the formation of the desired highly-porous foams. Various high-shear mixers are known in the art and believed suitable for use with example aspects of the present disclosure. High-shear mixers typically employ a tank holding the foam precursor and/or one or more pipes through which the foam precursor is directed. The high-shear mixers may use a series of screens and/or rotors to work the precursor and cause aggressive mixing of the components and air. In a particular example embodiment, the first tank 14 and/or the second tank 26 is provided having therein one or more rotors or impellors and associated stators. The rotors or impellors are rotated at high speeds in order to cause flow and shear. Air may, for example, be introduced into the tank at various positions or simply drawn in by the action of the mixers 24, 34. While the specific mixer design may influence the speeds necessary to achieve the desired mixing and shear, in certain example embodiments suitable rotor speeds may be greater than about five hundred rotations per minute (500 rpm) and, for example, be between about one thousand rotations per minute (1000 rpm) and about six thousand rotations per minute (6000 rpm) or between about two thousand rotations per minute (2000 rpm) and about four thousand rotations per minute (4000 rpm). In certain example embodiments, with respect to rotor based high-shear mixers, the mixer(s) 24, 34 may be run with the foam until the disappearance of the vortex in the foam or a sufficient volume increase is achieved.

In addition, it is noted the foaming process may be accomplished in a single foam generation step or in sequential foam generation steps for the first tank 14 and/or the second tank 26. For example, in one example embodiment, all of the components of the first fluid supply 16 in the first tank 14 (e.g., the supply of the fluid 18, fibers 20, and surfactant 22) may be mixed together to form a slurry from which a foam is formed. Alternatively, one or more of the individual components may be added to the foaming fluid, an initial mixture formed (e.g., a dispersion or foam), after which the remaining components may be added to the initially foamed slurry and then all of the components acted upon to form the final foam. In this regard, the fluid 18 and surfactant 22 may be initially mixed and acted upon to form an initial foam prior to the addition of any solids. Fibers, if desired, may then be added to the water/surfactant foam and then further acted upon to form the final foam. As a further example alternative, the fluid 18 and fibers 20, such as a high-density cellulose pulp sheet, may be aggressively mixed at a higher consistency to form an initial dispersion after which the foaming surfactant, additional water and other components, such as synthetic fibers, are added to form a second mixture which is then mixed and acted upon to form the foam.

The foam density of the foam forming the first fluid supply 16 in the first tank 14 and/or the foam forming the second fluid supply 28 in the second tank 26 may vary depending upon the particular application and various factors, such as the fiber stock used. In some example implementations, the foam density of the foam may be greater than about one hundred grams per liter (100 g/L), such as greater than about two hundred and fifty grams per liter (250 g/L), such as greater than about three hundred grams per liter (300 g/L). The foam density may be generally less than about eight hundred grams per liter (800 g/L), such as less than about five hundred grams per liter (500 g/L), such as less than about four hundred grams per liter (400 g/L), such as less than about three hundred and fifty grams per liter (350 g/L). In some example implementations, a lower density foam may be used having a foam density of generally less than about three hundred and fifty grams per liter (350 g/L), such as less than about three hundred and forty grams per liter (340 g/L), such as less than about three hundred and thirty grams per liter (330 g/L).

In some example embodiments, the apparatus 10 may also include a first pump 36 and a second pump 38. The first pump 36 may be in fluid communication with the first fluid supply 16 and may be configured for pumping the first fluid supply 16 to transfer the first fluid supply 16. The second pump 38 may be in fluid communication with the second fluid supply 28 and may be configured for pumping the second fluid supply 28 to transfer the second fluid supply 28. In some example embodiments, the first pump 36 and/or the second pump 38 may be a progressive cavity pump or a centrifugal pump; however, it is contemplated that other suitable types of pumps may be used. Additionally, as discussed further below, in some example embodiments, the apparatus may be provided with a single pump that may pump a single fluid supply into a first fluid supply 16 and a second fluid supply 28.

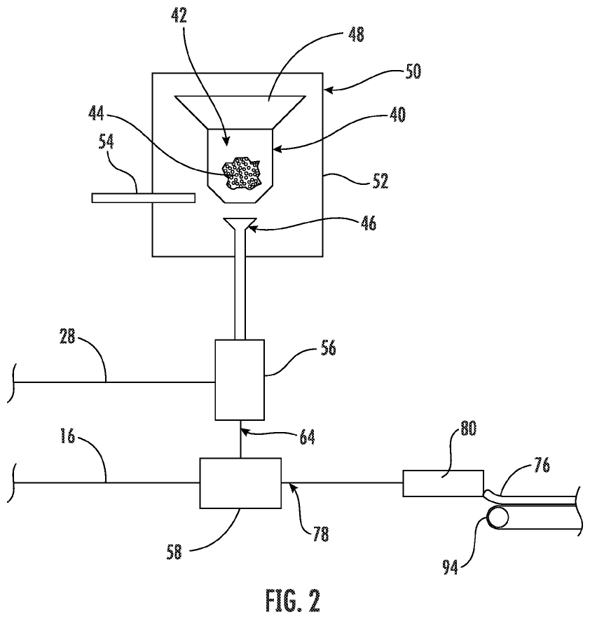

As depicted in FIGS. 1 and 2, the apparatus 10 may also optionally include a component feed system 40. The component feed system 40, for instance, may be configured to feed a particulate material into the fiber furnish, such as superabsorbent particles. When forming a substrate solely from fibers, however, the component feed system 40 may not be needed, including use of the second fluid supply 28. When present, the component feed system 40 may include a component supply area 42 for receiving a supply of a component 44 as shown in the partial cut-away portion of the component supply area 42 illustrated in FIG. 2. The component feed system 40 may also include an outlet conduit 46. The outlet conduit 46 may be circular in cross-sectional shape or may be configured in a rectangular fashion, such as to form a slot. The component feed system 40 may also include a hopper 48. The hopper 48 may be coupled to the component supply area 42 and may be configured for refilling the supply of the component 44 to the component supply area 42.

In some example embodiments, the component feed system 40 may include a bulk solids pump. Some examples of bulk solids pumps that may be used herein include systems that utilize screws/augers, belts, vibratory trays, rotating discs, or other known systems for handling and discharging the supply of the component 44. Other types of feeders may be used for the component feed system 40, such as, for example, an ingredient feeder, such as those manufactured by Christy Machine & Conveyor, Fremont, Ohio. The component feed system 40 may also be configured as a conveyor system in some example embodiments.

The component feed system 40 may also include a fluid control system 50. The fluid control system 50 may be configured to control the gas entrainment into the fluid supply into which the supply of the component 44 is placed. In some example embodiments, the fluid control system 50 may include a housing 52. The housing 52 may form a pressurized seal volume around the component feed system 40. In other example embodiments, the fluid control system 50 may be formed as an integral part to the structure component feed system 40 itself, such that a separate housing 52 surrounding the component feed system 40 may not be required. As depicted in FIGS. 1 and 2, the fluid control system 50 may also include a bleed orifice 54 in some example embodiments.

The supply of the component 44 may be in the form of a particulate and/or a fiber. In one example embodiment as described herein, the supply of the component 44 may be superabsorbent material (SAM) in particulate form. In some example embodiments, SAM may be in the form of a fiber. Of course, other types of components, as described further below, are also contemplated as being utilized in the apparatus 10 and methods as described herein. The component feed system 40 as described herein may be particularly beneficial for a supply of component 44 that is most suitably maintained in a dry environment with minimal of exposure to fluid or foam utilized in the apparatus 10 and methods described herein.

In some example embodiments, the apparatus 10 and methods described herein may include a first mixing junction 56 and a second mixing junction 58. In preferred example embodiments, the first mixing junction 56 may be an eductor. The first mixing junction 56 may be in fluid communication with the outlet conduit 46 of the component feed system 40 and in fluid communication with the second fluid supply 28. The first mixing junction 56 may be configured as a co-axial eductor. The first mixing junction 56 may mix the supply of the component 44 from the component feed system 40 with the second fluid supply 28.

Referring back to FIG. 1, the apparatus 10 may include a second mixing junction 58 in some example embodiments. The second mixing junction 58 may be configured for mixing the second fluid supply 28, including the component from the supply of the component 44, with the first fluid supply 16. As the second fluid supply 28, including the component from the supply of the component 44, exits the discharge 64 of the first mixing junction 56, the second fluid supply 28 may be transferred to the second mixing junction 58. The first fluid supply 16 may be delivered to the second mixing junction 58 by the first pump 36. The second mixing junction 58 may mix the first fluid supply 16 and any of the components of the first fluid supply 16 (e.g., fluid 18, fibers 20, surfactant 22) with the second fluid supply 28 and any of the components of the second fluid supply 28 (e.g., fluid 30, surfactant 32) and the component from the supply of the component 44 to provide a resultant slurry 76. The resultant slurry 76 may be transferred from the second mixing junction 58 through a discharge 78 of the second mixing junction 58 and to a headbox 80.

The headbox 80 may be provided to transfer the resultant slurry 76 to form a substrate 12. The headbox 80 may have a machine direction 81 and a cross direction 83. The machine direction 81 may be in the direction of the transfer of the resultant slurry 76 through the headbox 80. From the headbox 80, the wet slurry 76 may be deposited onto a forming surface 94 made in accordance with example aspects of the present disclosure.

In the example embodiment illustrated in FIG. 1, the forming surface 94 is shown in a horizonal position. In alternative example embodiments, the forming surface 94 may be placed or oriented at an angle to the headbox 80 such that the slurry 76 moves in an upward motion once deposited onto the forming surface 94. The forming surface 94, for instance, may be positioned at an angle to the horizontal of greater than about five degrees (5°), such as greater than about ten degrees (10°), such as greater than about fifteen degrees (15°), and generally less than about fifty degrees (50°), such as less than about forty degrees (40°), such as less than about thirty degrees (30°), such as less than about twenty degrees (20°). The headbox 80 may be designed to produce single layer substrates or may be used to produce multi-layer substrates. Substrates 12 may be made according to example aspects of the present disclosure, for instance, having two (2) layers, three (3) layers, four (4) layers, five (5) layers, or the like. Each layer may have a different fiber furnish if desired.

Once the slurry 76 is deposited onto the forming surface 94, fluids may be removed from the suspension for forming a nonwoven substrate. In this regard, the forming surface 94 may be a foraminous sheet that is porous and may at least in part be made from a woven belt or screen. The system may further include a dewatering system 96 that may be configured to remove liquids from the slurry 76 through the forming surface 94. The dewatering system 96, for instance, may provide a vacuum to the slurry 76 to pull fluids from the slurry for forming a consolidated substrate 12.

As shown in FIG. 1, the process may also include a drying system 98. The drying system 98 may be configured to dry the newly formed nonwoven substrate 12. In general, any suitable dryer may be incorporated into the drying system 98. The drying system 98, for instance, may be a through-air dryer, one or a series of heated drums, an oven, or the like. The process may further include a winding system 99 that may be configured to wind the substrate 12 after drying into a roll.

As described above, the nonwoven substrates made according to example aspects of the present disclosure may be formed according to a foam forming process. The foam forming process may provide various advantages. Foam forming processes, for instance, may more easily process longer fibers, require less water to form the webs, and may require less energy to dry the webs.

When forming the nonwoven substrate according to a foam forming process, the wet slurry 76 may be a foamed suspension of fibers alone or in combination with other solid materials. In some example embodiments, the foaming fluid may include between about eighty five percent (85%) to about ninety-nine and ninety-nine hundredths percent (99.99%) of the foam (by weight). In some example embodiments, the foaming fluid used to make the foam may include at least about eighty-five percent (85%) of the foam (by weight). In certain example embodiments, the foaming fluid may include between about ninety percent (90%) and about ninety-nine and ninety-nine hundredths percent (99.9%) of the foam (by weight). In certain other example embodiments, the foaming fluid may include between about ninety-three percent (93%) and ninety-nine and a half percent (99.5%) of the foam or even between about ninety-five percent (95%) and about ninety-nine percent (99%) of the foam (by weight). In preferred example embodiments, the foaming fluid may be water; however, it is contemplated that other processes may utilize other foaming fluids.

The foam forming processes as described herein may utilize one or more foaming agents, such as surfactants, in order to form the foam. The fibers and surfactant, together with the foaming liquid and any additional components, may form a stable dispersion capable of substantially retaining a high degree of porosity for longer than the drying process. In this regard, the surfactant may be selected so as to provide a foam having a foam half-life of at least two minutes (2 min), more desirably at least five minutes (5 min), and most desirably at least ten minutes (10 min). A foam half-life may be a function of surfactant types, surfactant concentrations, foam compositions/solid level, and mixing power/air content in a foam. The foaming surfactant used in the foam may be selected from one or more known in the art that are capable of providing the desired degree of foam stability. In this regard, the foaming surfactant may be selected from anionic, cationic, nonionic and amphoteric surfactants provided the foaming surfactant, alone or in combination with other components, provide the necessary foam stability, or foam half-life. As will be appreciated, more than one surfactant may be used, including different types of surfactants, as long as the surfactants are compatible, and more than one surfactant of the same type. For example, a combination of a cationic surfactant and a nonionic surfactant or a combination of an anionic surfactant and a nonionic surfactant may be used in some example embodiments due to compatibilities between the surfactants. However, in some example embodiments, a combination of a cationic surfactant and an anionic surfactant may not be satisfactory to combine due to incompatibilities between the surfactants.

Anionic surfactants believed suitable for use with example aspects of the present disclosure include, without limitation, anionic sulfate surfactants, alkyl ether sulfonates, alkylaryl sulfonates, or mixtures or combinations thereof. Examples of alkylaryl sulfonates include, without limitation, alkyl benzene sulfonic acids and their salts, dialkylbenzene disulfonic acids and their salts, dialkylbenzene sulfonic acids and their salts, alkylphenol sulfonic acids/condensed alkylphenol sulfonic acids and their salts, or mixture or combinations thereof. Examples of additional anionic surfactants believed suitable for use in example aspects of the present disclosure include alkali metal sulforicinates, sulfonated glyceryl esters of fatty acids, such as sulfonated monoglycerides of coconut oil acids, salts of sulfonated monovalent alcohol esters, such as sodium oleylisethianate, metal soaps of fatty acids, amides of amino sulfonic acids, such as the sodium salt of oleyl methyl tauride, sulfonated products of fatty acids nitriles, such as palmitonitrile sulfonate, alkali metal alkyl sulfates, such as sodium lauryl sulfate, ammonium lauryl sulfate or triethanolamine lauryl sulfate, ether sulfates having alkyl groups of eight (8) or more carbon atoms, such as sodium lauryl ether sulfate, ammonium lauryl ether sulfate, sodium alkyl aryl ether sulfates, and ammonium alkyl aryl ether sulfates, sulphuric esters of polyoxyethylene alkyl ether, sodium salts, potassium salts, and amine salts of alkylnapthylsulfonic acid. Certain phosphate surfactants including phosphate esters, such as sodium lauryl phosphate esters or those available from the Dow Chemical Company under the tradename TRITON are also believed suitable for use herewith. A particularly desired anionic surfactant is sodium dodecyl sulfate (SDS).

Cationic surfactants are also believed suitable for use with example aspects of the present disclosure for manufacturing some example embodiments of substrates. In some example embodiments, such as those including superabsorbent material, cationic surfactants may be less preferable to use due to potential interaction between the cationic surfactant(s) and the superabsorbent material, which may be anionic. Foaming cationic surfactants include, without limitation, monocarbyl ammonium salts, dicarbyl ammonium salts, tricarbyl ammonium salts, monocarbyl phosphonium salts, dicarbyl phosphonium salts, tricarbyl phosphonium salts, carbylcarboxy salts, quaternary ammonium salts, imidazolines, ethoxylated amines, quaternary phospholipids and so forth. Examples of additional cationic surfactants include various fatty acid amines and amides and their derivatives, and the salts of the fatty acid amines and amides. Examples of aliphatic fatty acid amines include dodecylamine acetate, octadecylamine acetate, and acetates of the amines of tallow fatty acids, homologues of aromatic amines having fatty acids, such as dodecylanalin, fatty amides derived from aliphatic diamines, such as undecylimidazoline, fatty amides derived from aliphatic diamines, such as undecylimidazoline, fatty amides derived from disubstituted amines, such as oleylaminodiethylamine, derivatives of ethylene diamine, quaternary ammonium compounds and their salts which are exemplified by tallow trimethyl ammonium chloride, dioctadecyldimethyl ammonium chloride, didodecyldimethyl ammonium chloride, dihexadecyl ammonium chloride, alkyltrimethylammonium hydroxides, dioctadecyldimethylammonium hydroxide, tallow trimethylammonium hydroxide, trimethylammonium hydroxide, methylpolyoxyethylene cocoammonium chloride, and dipalmityl hydroxyethylammonium methosulfate, amide derivatives of amino alcohols, such as beta-hydroxylethylstearylamide, and amine salts of long chain fatty acids. Further examples of cationic surfactants believed suitable for use with example aspects of the present disclosure include benzalkonium chloride, benzethonium chloride, cetrimonium bromide, distearyldimethylammonium chloride, tetramethylammonium hydroxide, and so forth.

Nonionic surfactants believed suitable for use in example aspects of the present disclosure include, without limitation, condensates of ethylene oxide with a long chain fatty alcohol or fatty acid, condensates of ethylene oxide with an amine or an amide, condensation products of ethylene and propylene oxides, fatty acid alkylol amide and fatty amine oxides. Various additional examples of non-ionic surfactants include alkyl polyglycoside, stearyl alcohol, sorbitan monostearate, octyl glucoside, octaethylene glycol monododecyl ether, lauryl glucoside, cetyl alcohol, cocamide MEA, monolaurin, polyoxyalkylene alkyl ethers, such as polyethylene glycol long chain (12-14 C) alkyl ether, polyoxyalkylene sorbitan ethers, polyoxyalkylene alkoxylate esters, polyoxyalkylene alkylphenol ethers, ethylene glycol propylene glycol copolymers, polyvinyl alcohol, alkylpolysaccharides, polyethylene glycol sorbitan monooleate, octylphenol ethylene oxide, and so forth.

The foaming surfactant may be used in varying amounts as necessary to achieve the desired foam stability and air-content in the foam. In certain example embodiments, the foaming surfactant may include between about five-thousandths percent (0.005%) and about five percent (5%) of the foam (by weight). In certain example embodiments the foaming surfactant may include between about five-hundredths percent (0.05%) and about three percent (3%) of the foam or even between about five-hundredths percent (0.05%) and about two percent (2%) of the foam (by weight).

The fibers and/or other solid material used to form nonwoven substrates in accordance with example aspects of the present disclosure may vary depending upon the particular application and the desired result. For instance, the nonwoven substrates may be formed from pulp fibers, other natural fibers such as cotton fibers, regenerated cellulose fibers, synthetic polymer fibers, binder fibers, superabsorbent particles, and mixtures thereof. When producing tissue products, for instance, the nonwoven substrate may contain pulp fibers in an amount greater than about ninety percent (90%) by weight. When producing wiping products, the nonwoven substrate may be made from pulp fibers alone or in combination with polymer synthetic fibers, such as polyester fibers. When producing nonwoven substrates for use in absorbent articles, the nonwoven substrate may include superabsorbent particles combined with at least one type of fiber. The at least one type of fiber may include pulp fibers, pulp fibers combined with polymer synthetic fibers, pulp fibers combined with binder fibers, pulp fibers combined with synthetic polymer fibers and binder fibers, pulp fibers combined with regenerated cellulose fibers, or any other suitable mixtures.

A wide variety of cellulosic fibers are believed suitable for use herein. In some example embodiments, the fibers utilized may be conventional papermaking fibers, such as wood pulp fibers formed by a variety of pulping processes, such as kraft pulp, sulfite pulp, bleached chemithermomechanical pulp (BCTMP), chemithermomechanical pulp (CTMP), pressure/pressure thermomechanical pulp (PTMP), thermomechanical pulp (TMP), thermomechanical chemical pulp (TMCP), and so forth. By way of example only, fibers and methods of making wood pulp fibers are disclosed in U.S. Pat. No. 4,793,898 to Laamanen et al.; U.S. Pat. No. 4,594,130 to Chang et al.; U.S. Pat. No. 3,585,104 to Kleinhart; U.S. Pat. No. 5,595,628 to Gordon et al.; U.S. Pat. No. 5,522,967 to Shet; and so forth. Further, the fibers may be any high-average fiber length wood pulp, low-average fiber length wood pulp, or mixtures of the same. Examples of suitable high-average length pulp fibers include softwood fibers, such as, but not limited to, northern softwood, southern softwood, redwood, red cedar, hemlock, pine (e.g., southern pines), spruce (e.g., black spruce), and the like. Examples of suitable low-average length pulp fibers include hardwood fibers, such as, but not limited to, Eucalyptus, maple, birch, aspen, and the like.

Moreover, if desired, secondary fibers obtained from recycled materials may be used, such as fiber pulp from sources, such as, for example, newsprint, reclaimed paperboard, and office waste. In a particularly preferred example embodiment, refined fibers are utilized in the tissue web such that the total amount of virgin and/or high average fiber length wood fibers, such as softwood fibers, may be reduced.

Regardless of the origin of the wood pulp fiber, the wood pulp fibers may preferably have an average fiber length greater than about two-tenths millimeter (0.2 mm) and less than about three millimeters (3 mm), such as from about thirty-five hundredths millimeter (0.35 mm) and about to and a half millimeter (2.5 mm), or between about a half millimeter (0.5 mm) to about two millimeters (2 mm) or even between about seven-tenths millimeter (0.7 mm) and about one and a half millimeters (1.5 mm).

In addition, other cellulosic fibers that may be used in example aspects of the present disclosure include nonwoody fibers. As used herein, the term “non-wood fiber” generally refers to cellulosic fibers derived from non-woody monocotyledonous or dicotyledonous plant stems. Non-limiting examples of dicotyledonous plants that may be used to yield non-wood fiber include kenaf, jute, flax, ramie and hemp. Non-limiting examples of monocotyledonous plants that may be used to yield non-wood fiber include cereal straws (wheat, rye, barley, oat, etc.), stalks (corn, cotton, sorghum, Hesperaloe funifera, etc.), canes (bamboo, sisal, bagasse, etc.) and grasses (Miscanthus, esparto, lemon, sabai, switchgrass, etc.). In still other certain instances, non-wood fiber may be derived from aquatic plants, such as water hyacinth, microalgae, such as Spirulina, and macroalgae seaweeds, such as red or brown algae.

Still further, other cellulosic fibers for making substrates herein may include synthetic cellulose fiber types formed by spinning, including rayon in all its varieties, and other fibers derived from viscose or chemically-modified cellulose such as, for example, those available under the trade names LYOCELL and TENCEL.

In some example embodiments, the non-woody and synthetic cellulosic fibers may have fiber length greater than about two-tenths millimeter (0.2 mm) including, for example, having an average fiber size between about a half millimeter (0.5 mm) and about thirty millimeters (30 mm) or between about seventy-five hundredths millimeter (0.75 mm) and about eight millimeters (8 mm) or even between about one millimeter (1 mm) and about five millimeters (5 mm).

Additional fibers that may be utilized in example aspects of the present disclosure include synthetic polymer fibers. By way of non-limiting example, such fibers may include polyolefin, polyester (PET), polyamide, polylactic acid, or other fiber forming polymers. Polyolefin fibers, such as polyethylene (PE) and polypropylene (PP), are particularly well suited for use in example aspects of the present disclosure.

The synthetic polymer fibers may have fiber length greater than about two-tenths millimeter (0.2 mm) including, for example, having an average fiber size between about a half millimeter (0.5 mm) and about fifty millimeters (50 mm) or between about seventy-five hundredths millimeter (0.75) and about ten millimeters (10 mm) or between about one millimeter (1 mm) and about eight millimeters (8 mm). In one example embodiment, the synthetic polymer fibers may be staple fibers having a fiber length of greater than about two millimeters (2 mm), such as greater than about three millimeters (3 mm), such as greater than about four millimeters (4 mm), and generally less than about eight millimeters (8 mm), such as less than about seven millimeters (7 mm), such as less than about six millimeters (6 mm), such as less than about five millimeters (5 mm), such as less than about four millimeters (4 mm). The polymer fibers may have a size of less than about five (5) denier, such as less than about three (3) denier, such as less than about two (2) denier, and greater than about a half (0.5) denier, such as greater than about one (1) denier.

In one example embodiment, the fiber furnish may include a binder material that consolidates the substrate once activated. For instance, the fiber furnish may include binder fibers that bond to adjacent fibers when heated, such as during the drying of the substrate.

Binder materials that may be used in example aspects of the present disclosure may include, but are not limited to, thermoplastic binder fibers, such as PET/PE bicomponent binder fiber, and water-compatible adhesives, such as latexes. In some example embodiments, binder materials as used herein may be in powder form, for example, such as thermoplastic PE powder. Importantly, the binder may include one that is water insoluble on the dried substrate. In certain example embodiments, latexes used in example aspects of the present disclosure may be cationic or anionic to facilitate application to and adherence to cellulosic fibers that may be used herein. For instance, latexes believed suitable for use include, but are not limited to, anionic styrene-butadiene copolymers, polyvinyl acetate homopolymers, vinyl-acetate ethylene copolymers, vinyl-acetate acrylic copolymers, ethylene-vinyl chloride copolymers, ethylene-vinyl chloride-vinyl acetate terpolymers, acrylic polyvinyl chloride polymers, acrylic polymers, nitrile polymers, as well as other suitable anionic latex polymers known in the art. Examples of such latexes are described in U.S. Pat. No. 4,785,030 to Hager, U.S. Pat. No. 6,462,159 to Hamada, U.S. Pat. No. 6,752,905 to Chuang et al. and so forth. Examples of suitable thermoplastic binder fibers include, but are not limited to, monocomponent and multi-component fibers having at least one relatively low melting thermoplastic polymer, such as polyethylene. In certain example embodiments, polyethylene/polypropylene sheath/core staple fibers may be used. Binder fibers may have lengths in line with those described herein above in relation to the synthetic polymer fibers.

Binders in liquid form, such as latex emulsions, may include between about zero percent (0%) and about ten percent (10%) by weight of the substrate. Binder fibers, when used, may be added proportionally to the other components to achieve the desired fiber ratios and structure. As an example, in some example embodiments, binder fibers may include between about zero percent (0%) and about fifty percent (50%) of the total fiber weight, and more preferably, between about five percent (5%) to about forty percent (40%) of the total fiber weight in some example embodiments.

One additional additive that may be added during the formation of the substrates 12 as described herein may be a superabsorbent materials (SAM). SAM is commonly provided in a particulate form and, in certain example aspects, may include polymers of unsaturated carboxylic acids or derivatives thereof. These polymers are often rendered water insoluble, but water swellable, by crosslinking the polymer with a di- or polyfunctional internal crosslinking agent. These internally cross-linked polymers are at least partially neutralized and commonly contain pendant anionic carboxyl groups on the polymer backbone that enable the polymer to absorb aqueous fluids, such as body fluids. Typically, the SAM particles are subjected to a post-treatment to crosslink the pendant anionic carboxyl groups on the surface of the particle. SAMs are manufactured by known polymerization techniques, desirably by polymerization in aqueous solution by gel polymerization. The products of this polymerization process are aqueous polymer gels, i.e., SAM hydrogels, that are reduced in size to small particles by mechanical forces, then dried using drying procedures and apparatus known in the art. The drying process is followed by pulverization of the resulting SAM particles to the desired particle size. Examples of superabsorbent materials include, but are not limited to, those described in U.S. Pat. No. 7,396,584 Azad et al., U.S. Pat. No. 7,935,860 Dodge et al., US2005/5245393 to Azad et al., US2014/09606 to Bergam et al., WO2008/027488 to Chang et al. and so forth. In addition, in order to aid processing, the SAM may be treated in order to render the material temporarily non-absorbing during the formation of the foam and formation of the highly-expanded foam. For example, in one example aspect, the SAM may be treated with a water-soluble protective coating having a rate of dissolution selected such that the SAM is not substantially exposed to the aqueous carrier until the highly-expanded foam has been formed and drying operations initiated. Alternatively, in order to prevent or limit premature expansion during processing, the SAM may be introduced into the process at low temperatures.

Superabsorbent materials may be incorporated into the nonwoven substrates generally in an amount from about one percent (1%) to about ninety-five percent (95%) by weight, including all increments of one percent (1%) by weight therebetween. For instance, the superabsorbent material may be present in the substrate in an amount greater than about ten percent (10%) by weight, such as in an amount greater than about twenty percent (20%) by weight, such as in an amount greater than about thirty percent (30%) by weight, such as in an amount greater than about forty percent (40%) by weight, such as in an amount greater than about fifty percent (50%) by weight, and generally in an amount less than about sixty-five percent (65%) by weight, such as in an amount less than about fifty-five percent (55%) by weight, such as in an amount less than about forty-five percent (45%) by weight.

Other additives that can be incorporated into the nonwoven substrates include wet strength additives, permanent wet strength additives, temporary wet strength additives, dry strength additives, and the like.

Still other additional components that may be added to the substrates include one or more pigments, opacifying agents, anti-microbial agents, pH modifiers, skin benefit agents, odor absorbing agents, fragrances, thermally expandable microspheres, foam particles (such as, pulverized foam particles), and so forth as desired to impart or improve one or more physical or aesthetic attributes. In certain example embodiments, the substrates may include skin benefit agents, such as antioxidants, astringents, conditioners, emollients, deodorants, external analgesics, film formers, humectants, hydrotropes, pH modifiers, surface modifiers, skin protectants, and so forth. When employed, miscellaneous components desirably include less than about two percent (2%) of the substrate (by weight).

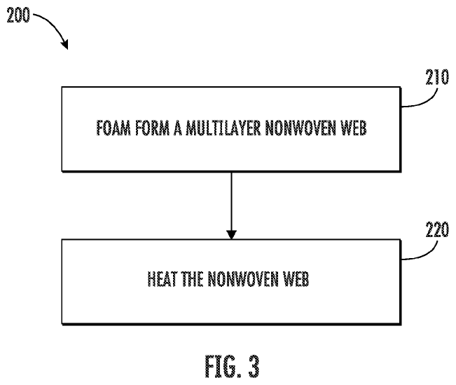

FIG. 3 illustrates a method 200 for forming a nonwoven material according to an example embodiment of the present subject matter. Method 200 may be performed with in apparatus 10 as part of a foam forming process for a nonwoven material, such as substrate 12. Thus, method 200 is described in greater detail below in the context of apparatus 10. However, it will be understood that method 200 may be performed or implemented in any suitable foam forming system in alternative example embodiments. Utilizing method 200, a nonwoven material, such as substrate 12, may be formed with formed with three-dimensional topography, e.g., on an outer surface of one of the layers of substrate 12.

At 210, substrate 12 may be formed with a plurality of layers. For example, headbox 80 may deposit multiple slurries 76 onto forming surface 94 to form substrate 12 with multiple layers, such as two (2) layers, three (3) layers, four (4) layers, five (5) layers, or more, at 210. At 210, foam forming substrate 12 may include depositing a first foam and a second foam onto forming surface 94 with head box 80.

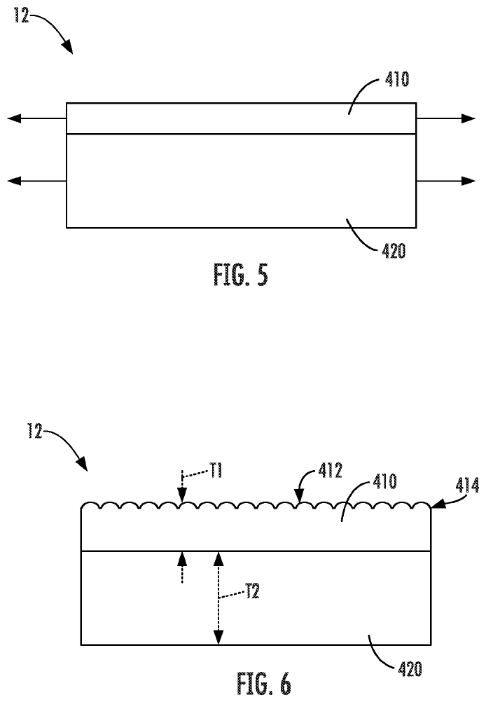

Each layer in substrate 12 may have a different fiber furnish in certain example embodiments. For example, as shown in FIGS. 5 and 6, substrate 12 may include a first layer 410 and a second layer 420. First layer 410 may be configured as an acquisition layer, and second layer 420 may be configured as an absorbent layer. Thus, first layer 410 may be configured for collecting and moving away from the body feces and other bodily fluids, e.g., towards or to second layer 420 that is configured for collecting the body feces and other bodily fluids therein. First and second layers 410, 420 may have different fiber furnishes in certain example embodiments. For instance, second layer 420 may include superabsorbent materials, e.g., in an amount from about one percent (1%) to about ninety-five percent (95%) by weight. Conversely, first layer 410 may be substantially free of superabsorbent materials. As another example, second layer 420 may be substantially free of PET fibers, and first layer 410 include PET fibers. Thus, e.g., the first foam may include water and a plurality of first binder fibers, such as PET/PE fibers (e.g., with PET as the core and PE as the sheath), and the second foam may include water and a plurality of second binder fibers, such as PET/PE fibers (e.g., with different fiber diameters and/or length than the PET/PE fibers of the first binder fibers). The difference in fiber furnishes between the first and second layers 410, 420 may facilitate differential shrinkage between the first and second layers 410, 420 as discussed in greater detail below.

In certain example embodiments, a basis weight 410 of the first layer may be selected to assist with formation of three-dimensional topography on first layer 410. For instance, the first layer 410 may generally have a basis weight of greater than about 40 gsm, such as greater than about 50 gsm, such as greater than about 60 gsm, such as greater than about 70 gsm, such as greater than about 80 gsm, such as greater than about 90 gsm, such as greater than about 100 gsm. The basis weight of the first layer 410 may generally be less than about 200 gsm, such as less than about 150 gsm, such as less than about 100 gsm.

As shown in FIGS. 5 and 6, first layer 410 and second layer 420 may also have different dimensional characteristics. For instance, each of first layer 410 and second layer 420 may have a respective thickness, e.g., perpendicular to both the machine direction 81 and the cross direction 83. Moreover, first layer 410 may have a thickness T1, and second layer 420 may have a thickness T2. The thickness T2 of the second layer 420 may be different than the thickness T1 of the first layer 410. For instance, the thickness T2 of the second layer 420 may greater than the thickness T1 of the first layer 410 in certain example embodiments. For example, the thickness T2 of the second layer 420 may be no less than one and a half times (1.5×), such as no less than two times (2×), such as no less than three times (3×), such as no less than five times (5×), such as no less than ten times (10×), greater than the thickness T1 of the first layer 410. Thus, e.g., the thickness T2 of the second layer 420 may be substantially greater than the thickness T1 of the first layer 410. Such relative dimensional differences between the first and second layers 410, 420 may facilitate differential shrinkage between the first and second layers 410, 420 as discussed in greater detail below.

At 220, substrate 12 may be cured such that second layer 420 contracts less than first layer 410. Such differential shrinkage may deform first layer 410 to produce a plurality of projections 412 at an outer surface 414 of first layer 410, e.g., that faces away from second layer 420. For instance, drying system 98 may be configured to at least partially dry substrate 12 at 220. In certain example embodiments, first layer 410 may be exposed to or placed in contact with a heated drum, an oven, or the like such that first layer 410 cures or dries more quickly than second layer 420 during 220. Thus, e.g., first layer 410 may face a heating element within an oven, contact a surface of a heated drum, or be exposed to a flow of heated air in a through drying system, such that first layer 410 shrinks to a greater degree than second layer 420 at 220. The relative dimensional differences between the first and second layers 410, 420 may also facilitate differential shrinkage between first and second layers 410, 420. Moreover, when second layer 420 is thicker than first layer 410, first layer 410 may cure or dry more quickly than second layer 420 in drying system 98 during 220. The difference in fiber furnishes between the first and second layers 410, 420 may also facilitate differential shrinkage between first and second layers 410, 420. Moreover, when second layer 420 includes superabsorbent materials and first layer 410 is substantially free of superabsorbent materials, first layer 410 may cure or dry more quickly than second layer 420 in drying system 98 at 220. Thus, at 220, the relative dimensional differences between the first and second layers 410, 420 and/or the difference in fiber furnishes between the first and second layers 410, 420 may facilitate differential shrinkage between first layer 410 and second layer 420.

It will be understood that in addition to the at least partial curing of the first layer, material selection between the first and second layers can also yield differential shrinkage between the two layers. For instance, incorporating PET fibers into the second layer 420 can cause the second layer 420 to shrink significantly more than the first layer 410 (e.g., without PLA fibers) during drying and curing such that decreasing the tension in the nonwoven material may cause small, local buckling of the first layer 410 to create resilient bumps on the first layer 410. As another example, second layer 420 may include a fiber capable of hydrogen bonding. In such example embodiments, differential shrinkage between first layer 410 and second layer 420 may be due at least in part to capillary condensation and hydrogen bonding during drying. In another example embodiment, second layer 420 may include superabsorbent particles that partially swell during processing in the wet end of the foam forming process. In such example embodiments, differential shrinkage between first layer 410 and second layer 420 may be due at least in part to shrinkage of the superabsorbent particles as water is removed. In another example embodiment, second layer 420 may include a fiber or other material that shrinks upon heating, such as PLA staple fibers. In such example embodiments, differential shrinkage between first layer 410 and second layer 420 during heating may be due at least in part to shrinkage of the heat-shrinkable material. Any suitable combination of mechanisms described above may also be utilized to provide differential shrinkage between first layer 410 and second layer 420.

As a particular example, a thermo-shrinkable bico fiber, such as PLA/co-PLA fibers commercially available from Trevira under the trade name as T-450, is capable of shrinking at least 30% of its original length under heating. When used in the second layer 420 as a binder fiber, both the first and second layers 410, 420 may be dried and cured at the same time at a temperature less than the shrinking temperature of the T-450 bico fibers. Once formed, the multilayer nonwoven may be exposed to a temperature near or slightly above the curing temperature of the T-450 bico fibers to trigger the shrinkage of the second layer 420 to achieve the projections 412 on the outer surface 414 of the first layer 410. Other materials, which are shrinkable and can be triggered by temperature or other mechanism, may also be used to form projections 412.

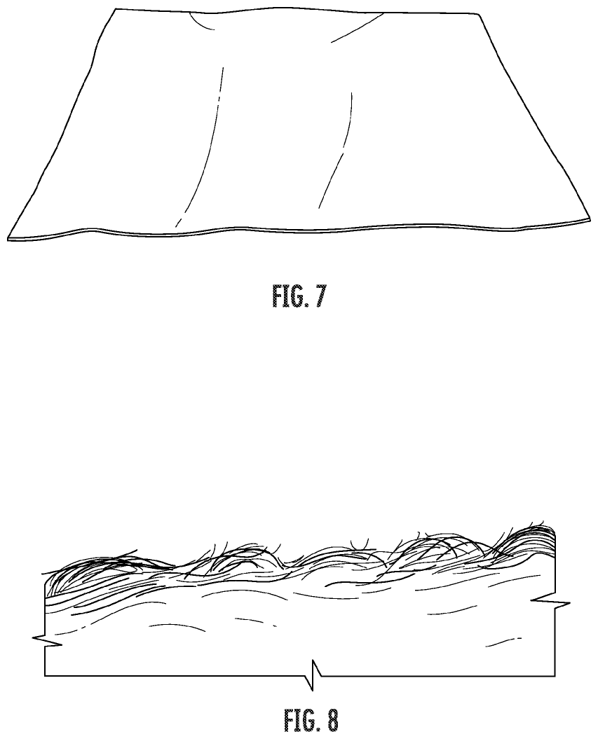

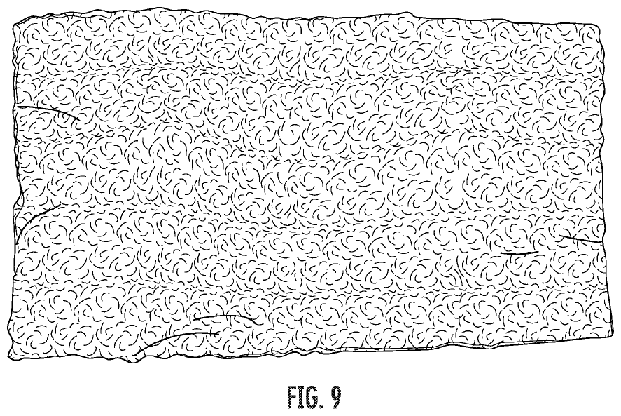

During the differential shrinkage between first layer 410 and second layer 420, small sections of the first layer 410 may buckle locally for form projections 412. The three-dimensional topography provided by projections 412 may advantageously increase void volume and increase compression resistance of first layer 410. Thus, first layer 410 may be configured for use as an acquisition layer in an absorbent articles, wiping product, or the like due to the projections 412 formed on first layer 410 during method 200 by differential shrinkage between first and second layers 410, 420. FIGS. 7 and 8, illustrate example three-dimensional topography provided by projections 412. As shown, projections 412 may be rounded domes formed by first layer 410, e.g., under relatively low tension. FIG. 9 illustrates another example three-dimensional topography provided by projections 412. As shown, projections 412 may be rounded ripples, e.g., formed under relatively high tension. The ripples may be elongated in the machine direction 81 on first layer 410.

Each of projections 412 may have a height, e.g., perpendicular to both the machine direction 81 and the cross direction 83. The height of projections 412 may be generally greater than about a half millimeter (0.5 mm), such as greater than about one millimeter (1 mm), such as greater than about one and a half millimeter (1.5 mm), such as greater than about two millimeter (2 mm), such as greater than about five millimeters (5 mm). The height of projections 412 may be generally less than about fifteen millimeters (15 mm), such as less than about ten millimeters (10 mm), such as less than about seven millimeters (7 mm), such as less than about five millimeters (5 mm), such as less than about three millimeters (3 mm). It will be understood that the above recited heights may correspond to an average height of the projections 412 on outer surface 414 and that individual heights of the projections 412 may vary (e.g., be less than or greater than) the values recited above.

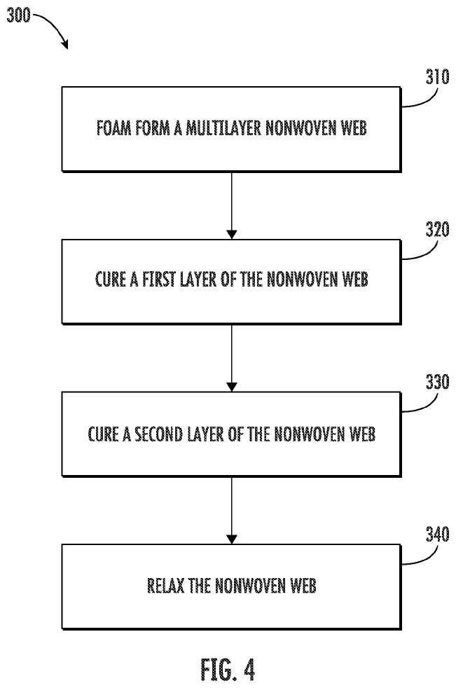

FIG. 4 illustrates a method 300 for forming a nonwoven material according to an example embodiment of the present subject matter. Method 300 may be performed with in apparatus 10 as part of a foam forming process for a nonwoven material, such as substrate 12. Thus, method 300 is described in greater detail below in the context of apparatus 10. However, it will be understood that method 300 may be performed or implemented in any suitable foam forming system in alternative example embodiments. Utilizing method 300, a nonwoven material, such as substrate 12, may be formed with formed with three-dimensional topography, e.g., on an outer surface of one of the layers of substrate 12.

At 310, substrate 12 may be formed with a plurality of layers. For example, headbox 80 may deposit multiple slurries 76 onto forming surface 94 to form substrate 12 with multiple layers, such as two (2) layers, three (3) layers, four (4) layers, five (5) layers, or more, at 310. At 310, foam forming substrate 12 may include depositing a first foam and a second foam onto forming surface 94 with head box 80.

Each layer in substrate 12 may have a different fiber furnish in certain example embodiments. For example, as described above and shown in FIGS. 5 and 6, substrate 12 may include a first layer 410 and a second layer 420. First layer 410 may be configured as an acquisition layer, and second layer 420 may be configured as an absorbent layer. Thus, first layer 410 may be configured for collecting and moving away from the body feces and other bodily fluids, e.g., towards or to second layer 420 that is configured for collecting the body feces and other bodily fluids therein. First and second layers 410, 420 may have different fiber furnishes in certain example embodiments. For instance, second layer 420 may include superabsorbent materials, e.g., in an amount from about one percent (1%) to about seventy percent (70%) by weight. Conversely, first layer 410 may be substantially free of superabsorbent materials. As another example, second layer 420 may include PET fibers, and first layer 410 may be substantially free of PET fibers. In certain example embodiments, e.g., the first foam may include water and a plurality of first binder fibers, such as PE fibers, and the second foam may include water and a plurality of second binder fibers, such as PET fibers. The difference in fiber furnishes between the first and second layers 410, 420 may facilitate differential shrinkage between the first and second layers 410, 420 as discussed in greater detail below.

In certain example embodiments, a basis weight 410 of the first layer may be selected to assist with formation of three-dimensional topography on first layer 410. For instance, the first layer 410 may generally have a basis weight of greater than about 40 gsm, such as greater than about 50 gsm, such as greater than about 60 gsm, such as greater than about 70 gsm, such as greater than about 80 gsm, such as greater than about 90 gsm, such as greater than about 100 gsm. The basis weight of the first layer 410 may generally be less than about 200 gsm, such as less than about 150 gsm, such as less than about 100 gsm.

As shown in FIGS. 5 and 6, first layer 410 and second layer 420 may also have different dimensional characteristics. For instance, each of first layer 410 and second layer 420 may have a respective thickness, e.g., perpendicular to both the machine direction 81 and the cross direction 83. Moreover, first layer 410 may have a thickness T1, and second layer 420 may have a thickness T2. The thickness T2 of the second layer 420 may be different than the thickness T1 of the first layer 410. For instance, the thickness T2 of the second layer 420 may greater than the thickness T1 of the first layer 410 in certain example embodiments. For example, the thickness T2 of the second layer 420 may be no less than one and a half times (1.5×), such as no less than two times (2×), such as no less than three times (3×), such as no less than five times (5×), such as no less than ten times (10×), greater than the thickness T1 of the first layer 410. Thus, e.g., the thickness T2 of the second layer 420 may be substantially greater than the thickness T1 of the first layer 410. Such relative dimensional differences between the first and second layers 410, 420 may facilitate differential shrinkage between the first and second layers 410, 420 as discussed in greater detail below.

At 320, first layer 410 may be at least partially cured while substrate 12 is in tension. For instance, drying system 98 may be configured to at least partially dry first layer 410 at 320. Thus, a water content of first layer 410 may decrease during the curing of first layer 410 at 320. In certain example embodiments, first layer 410 may be exposed to or placed in contact with a heated drum, an oven, or the like such that first layer 410 cures or dries more quickly than second layer 420. Thus, e.g., first layer 410 may face a heating element within an oven, contact a surface of a heated drum, or be exposed to a flow of heated air in a through drying system, such that a cure or dry rate of first layer 410 is greater than the cure or dry rate for second layer 420. The relative dimensional differences between the first and second layers 410, 420 may also facilitate curing or drying of first layer 410 more quickly than second layer 420. Moreover, when second layer 420 is thicker than first layer 410, first layer 410 may cure or dry more quickly than second layer 420 in drying system 98. The difference in fiber furnishes between the first and second layers 410, 420 may also facilitate curing or drying of first layer 410 more quickly than second layer 420. Moreover, when second layer 420 includes superabsorbent materials and first layer 410 is substantially free of superabsorbent materials, first layer 410 may cure or dry more quickly than second layer 420 in drying system 98. Thus, at 320, the relative dimensional differences between the first and second layers 410, 420 and/or the difference in fiber furnishes between the first and second layers 410, 420 may facilitate curing of first layer 410, e.g., while the second layer 420 remains substantially uncured and/or undried. Thus, the water content in second layer 420 may be greater than the water content in first layer 410 after the curing of first layer 410 at 320.

In certain example embodiments, portions of first layer 410 may be cured while substrate 12 is in tension at 320. Thus, e.g., the cure or dry rate of first layer 410 at a first portion of first layer 410 may be different than the cure or dry rate of first layer 410 at a second portion of first layer 410. Moreover, the curing or drying pattern for first layer 410 at 320 may be shaped or selected to control differential shrinkage between first and second layers 410, 420 and thus the three-dimensional topography provided by projections 412.

At 330, after at least partially curing the first layer 410 at 320, second layer 420 may be cured while substrate 12 is in tension. For instance, drying system 98 may be configured to dry second layer 420 at 330. In certain example embodiments, second layer 420 may be exposed to or placed in contact with a heated drum, an oven, or the like such that second layer 420 cures or dries. Thus, e.g., second layer 420 may face a heating element within an oven, contact a surface of a heated drum, or be exposed to a flow of heated air in a through drying system.

As may be seen from the above, first layer 410 is at least partially cured at 320 and then second layer 420 is subsequently cured at 330. By first at least partially curing first layer 410, a relationship between first layer 410 and second layer 420 may be fixed at least in regions of substrate 12. Moreover, at least partially curing first layer 410 may be define the interlayer adhesion between first and second layers 410, 420 prior to subsequently curing second layer 420.

At 340, substrate 12 may be relaxed. During the relaxing of substrate 12 at 340, second layer 420 may contract, and first layer 410 may deform to produce a plurality of projections 412 at an outer surface 414 of first layer 410, e.g., that faces away from second layer 420. In FIG. 5, substrate 12 is shown under tension. As noted above, substrate 12 may be under tension during the curing of first and second layers 410, 420 at 310 and 320. Conversely, substrate 12 is shown in a relaxed state in FIG. 6 with projections 412 formed at first layer 410 due to differential shrinkage between first and second layers 410, 420 as substrate relaxes at 340. Relaxation of substrate 12 at 340 may be at least partially performed during the curing of second layer 420 at 330. As another example, relaxation of substrate 12 at 340 may be at least partially performed after curing of second layer 420 at 330.

During the relaxation of substrate 12 while curing the second layer 420, small sections of the first layer 410 may buckle locally for form projections 412. The three-dimensional topography provided by projections 412 may advantageously increase void volume and increase compression resistance of first layer 410. Thus, first layer 410 may be configured for use as an acquisition layer in an absorbent articles, wiping product, or the like due to the projections 412 formed on first layer 410 during method 300 by differential shrinkage between first and second layers 410, 420. FIGS. 7 and 8, illustrate example three-dimensional topography provided by projections 412. As shown, projections 412 may be rounded domes formed by first layer 410, e.g., under relatively low tension. FIG. 9 illustrates another example three-dimensional topography provided by projections 412. As shown, projections 412 may be rounded ripples, e.g., formed under relatively high tension. The ripples may be elongated in the machine direction 81 on first layer 410.

Each of projections 412 may have a height, e.g., perpendicular to both the machine direction 81 and the cross direction 83. The height of projections 412 may be generally greater than about a half millimeter (0.5 mm), such as greater than about one millimeter (1 mm), such as greater than about one and a half millimeter (1.5 mm), such as greater than about two millimeter (2 mm), such as greater than about five millimeters (5 mm). The height of projections 412 may be generally less than about fifteen millimeters (15 mm), such as less than about ten millimeters (10 mm), such as less than about seven millimeters (7 mm), such as less than about five millimeters (5 mm), such as less than about three millimeters (3 mm). It will be understood that the above recited heights may correspond to an average height of the projections 412 on outer surface 414 and that individual heights of the projections 412 may vary (e.g., be less than or greater than) the values recited above.

As may be seen from the above, the present subject matter may advantageously provide a high void-volume, resilient layer on a nonwoven material. Such layer may be particularly suitable for use as an acquisition layer in a diaper or wipe. In general, example aspects of the present subject matter control interlayer bonding and shrinkage between two layers of a nonwoven material to create three-dimensional topography in one of the layers using a combination of material selection and a paired drying or curing process. Thus, e.g., during formation while the nonwoven material is under tension, a top layer of a multilayer (e.g., bilayer) nonwoven material may be entirely cured or cured at targeted portions such that a relationship between the top layer and an underlying second layer is fixed in at least some areas. The second layer may then be cured and dried while the tension in the second layer is controlled such that shrinkage of the nonwoven material due to drying of the second layer is controlled. As the second layer dries, the second layer will contract, and decreasing the tension in the nonwoven material may cause small, local buckling of the first layer to create resilient bumps on the first layer. The bumps advantageously increase void volume and also increase resiliency of the first layer due to the domed shape of the bumps. Controlling bonding points in the interlayer adhesion can also adjust and/or optimize the three-dimensional topography, such as the localized buckling, for stiffness and fluid handling characteristics of the nonwoven material. Thus, e.g., the shape and/or distribution of the resilient bumps may be controlled by the bonding points in the interlayer adhesion between the first and second layers in the nonwoven material. In certain example aspects, an overall structural stiffness of the nonwoven material may advantageously be controlled due to the formation of three-dimensional topography without adding additional raw material, which would increase overall stiffness.

The present disclosure may be better understood with reference to the following examples.

### EXAMPLES

The following examples were produced in accordance with the present disclosure.

In Example No. 1, a bilayer nonwoven substrate was formed on a forming surface. A top layer of the nonwoven substrate was about 35% bicomponent binder fibers and 65% PET staple fibers by weight with a basis weight of about 40 gsm, and a bottom layer of the nonwoven substrate was about 30% bicomponent binder fibers and 40% crosslinked pulp fiber (essentially not hydrogen-bond capable) by weight and about 30% PET fibers by weight with a basis weight of about 75 gsm for the fibers of the second layer. The second layer also included superabsorbent particles with a basis weight of about 400 gsm. The nonwoven substrate was dried in two passes for a total length of 14 feet at a run speed of 6 feet per minute with a residence time of 120 seconds at a temperature of 270° F. for the first pass and 220° F. for the second pass. The estimated shrinkage of the nonwoven substrate was 5%.

In Example No. 2, a bilayer nonwoven substrate was formed on a forming surface. A top layer of the nonwoven substrate was about 20% bicomponent binder fibers and 80% PET staple fibers by weight with a basis weight of about 40 gsm, and a bottom layer of the nonwoven substrate was about 40% bicomponent binder fibers, 10% PET fibers, and 50% NBSK wood pulp (hydrogen-bond capable) by weight with a basis weight of about 20 gsm for the fibers of the second layer. The second layer also included superabsorbent particles with a basis weight of about 345 gsm. The nonwoven substrate was dried in one pass for a total length of 7 feet at a run speed of 3 feet per minute with a residence time of 120 seconds at a temperature of 294° F. The estimated shrinkage of the nonwoven substrate was 29%.

In Example No. 3, a bilayer nonwoven substrate was formed on a forming surface. A top layer of the nonwoven substrate was about 20% bicomponent binder fibers and 80% PET staple fibers by weight with a basis weight of about 60 gsm, and a bottom layer of the nonwoven substrate was about 40% bicomponent binder fibers, 10% PET fibers, and 50% NBSK wood pulp (hydrogen-bond capable) by weight with a basis weight of about 20 gsm for the fibers of the second layer. The second layer also included superabsorbent particles with a basis weight of about 400 gsm. The nonwoven substrate was dried in one pass for a total length of 7 feet at a run speed of 3 feet per minute with a residence time of 120 seconds at a temperature of 294° F. The estimated shrinkage of the nonwoven substrate was 31%.

In the third example nonwoven material, machine direction oriented three-dimensional topography was formed on the top layer of the nonwoven materials. The machine direction oriented three-dimensional topography was observed to depend upon sufficient shrinkage of the second layer, predominantly in cross direction due to greater tension in the machine direction, and also upon sufficient basis weight in the first layer.

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

### EXAMPLE EMBODIMENTS

First example embodiment: A method for forming a nonwoven material, includes: foam forming a nonwoven web with a plurality of layers; at least partially curing a first layer of the plurality of layers while the nonwoven web is in tension; after at least partially curing the first layer, curing a second layer of the plurality of layers while the nonwoven web is in tension; and relaxing the nonwoven web such that the second layer contracts and the first layer deforms to produce a plurality of projections at an outer surface of the first layer that faces away from the second layer.

Second example embodiment: The method of the first example embodiment, wherein foam forming the nonwoven web comprises depositing a first foam and a second foam onto a formation surface, the first foam including water and a plurality of first binder fibers, the second foam including water and a plurality of second binder fibers.

Third example embodiment: The method of either the first example embodiment or the second example embodiment, wherein the first layer comprises a plurality of first binder fibers, and the second layer comprises a plurality of second binder fibers and a plurality of superabsorbent particles.

Fourth example embodiment: The method of any one of the first through third example embodiments, wherein the water content in the second layer is greater than the water content in the first layer after the curing of the first layer.

Fifth example embodiment: The method of any one of the first through fourth example embodiments, wherein the first layer comprises a plurality of first binder fibers, the second layer comprises a plurality of second binder fibers, and the second binder fibers are different than the first binder fibers.

Sixth example embodiment: The method of the fifth example embodiment, wherein the second binder fibers comprise PET fibers.

Seventh example embodiment: The method of any one of the first through sixth example embodiments, wherein the projections comprise a plurality of rounded ripples.

Eighth example embodiment: The method of any one of the first through seventh example embodiments, wherein a void volume of the first layer increases as the nonwoven web relaxes.

Nineth example embodiment: The method of any one of the first through eighth example embodiments, wherein the first layer is at least partially adhered to the second layer during the curing of the first layer.

Tenth example embodiment: The method of any one of the first through nineth example embodiments, wherein a thickness of the second layer is greater than a thickness of the first layer.

Eleventh example embodiment: The method of any one of the first through tenth example embodiments, wherein: at least partially curing the first layer comprises heating the first layer, a water content in the first layer decreasing during the heating of the first layer; and curing the second layer comprises heating the second layer, a water content in the second layer decreasing during the heating of the second layer.

Twelfth example embodiment: The method of any one of the first through tenth example embodiments, wherein the nonwoven web is a two-layer nonwoven web, and the plurality of layers consists of only the first and second layers.

Thirteenth example embodiment: The method of any one of the first through tenth example embodiments, wherein at least partially curing the first layer comprises curing a first portion of the first layer at a rate different than a second portion of the first layer

Fourteenth example embodiment: A nonwoven article, comprising a nonwoven material manufactured according to the method of any one of the first through eleventh example embodiments.

Fifteenth example embodiment: A nonwoven material, comprising: a foam formed nonwoven web with a first layer and a second layer, wherein the first layer is shrunken relative to the second layer such that the first layer forms a plurality of projections at an outer surface of the first layer that faces away from the second layer.

Sixteenth example embodiment: The nonwoven material of the fifteenth example embodiment, wherein the first layer comprises a plurality of first binder fibers, and the second layer comprises a plurality of second binder fibers and a plurality of superabsorbent particles.

Seventeenth example embodiment: The nonwoven material of either the fifteenth example embodiment or the sixteenth example embodiment, wherein the first layer comprises a plurality of first binder fibers, the second layer comprises a plurality of second binder fibers, and the second binder fibers are different than the first binder fibers.

Eighteenth example embodiment: The nonwoven material of any one of the fifteenth through seventeenth example embodiments, wherein the second binder fibers comprise PET fibers.

Nineteenth example embodiment: The nonwoven material of any one of the fifteenth through seventeenth example embodiments, wherein the projections comprise a plurality of rounded ripples.

Twentieth example embodiment: The nonwoven material of any one of the fifteenth through nineteenth example embodiments, wherein a thickness of the second layer is greater than a thickness of the first layer.

Twenty-First example embodiment: An absorbent article, comprising the nonwoven material of any one of the fifteenth through twentieth example embodiments, wherein the outer surface of the first layer faces towards a wearer of the absorbent article when the absorbent article is worn by the wearer.

Twenty-Second example embodiment: A method for forming a nonwoven material, comprising: foam forming a nonwoven web with a plurality of layers; heating a first layer of the plurality of layers in the nonwoven web while the nonwoven web is in tension, a water content in the first layer decreasing during the heating of the first layer; after heating the first layer and while the nonwoven web is in tension, heating a second layer of the plurality of layers in the nonwoven web, a water content in the second layer decreasing during the heating of the second layer; and, while heating the second layer, relaxing the nonwoven web such that the first layer deforms with a plurality of projections at an outer surface of the first layer that faces away from the second layer.

Twenty-Third example embodiment: A method for forming a nonwoven material, comprising: foam forming a nonwoven web with a plurality of layers, a first layer and a second layer of the plurality of layers at least partially interconnected; and heating the nonwoven web such that the second layer contracts less than the first layer, the first layer deforming in order to produce a plurality of projections at an outer surface of the first layer that faces away from the second layer.

Twenty-Fourth example embodiment: The method of the twenty-third example embodiment, wherein foam forming the nonwoven web comprises depositing a first foam and a second foam onto a formation surface, the first foam including water and a plurality of first binder fibers, the second foam including water and a plurality of second binder fibers.

Twenty-Fifth example embodiment: The method of either the twenty-third example embodiment or the twenty-fourth example embodiment, wherein the first layer comprises a plurality of first binder fibers, and the second layer comprises a plurality of second binder fibers and a plurality of superabsorbent particles.

Twenty-Sixth example embodiment: The method of any one of the twenty-third through the twenty-fifth example embodiments, wherein the first layer comprises a plurality of first binder fibers, the second layer comprises a plurality of second binder fibers, and the second binder fibers are different than the first binder fibers.

Twenty-Seventh example embodiment: The method of the twenty-sixth example embodiment, wherein the second binder fibers comprise PET fibers.

Twenty-Eighth example embodiment: The method of any one of the twenty-third through the twenty-seventh example embodiments, wherein the projections comprise a plurality of rounded ripples.

Twenty-Nineth example embodiment: The method of any one of the twenty-third through the twenty-eighth example embodiments, wherein a void volume of the first layer increases as the nonwoven web relaxes.

Thirtieth example embodiment: The method of any one of the twenty-third through the twenty-nineth example embodiments, wherein a thickness of the second layer is greater than a thickness of the first layer.

Thirty-First example embodiment: The method of any one of the twenty-third through the thirtieth example embodiments, wherein the nonwoven web is a two-layer nonwoven web, and the plurality of layers consists of only the first and second layers.

Thirty-Second example embodiment: A nonwoven article, comprising a nonwoven material manufactured according to the method of any one of the twenty-third through the thirty-first example embodiments.

1. A method for forming a nonwoven material, comprising:
foam forming a nonwoven web with a plurality of layers;
at least partially curing a first layer of the plurality of layers while the nonwoven web is in tension;
after at least partially curing the first layer, curing a second layer of the plurality of layers while the nonwoven web is in tension; and
relaxing the nonwoven web such that the second layer contracts and the first layer deforms to produce a plurality of projections at an outer surface of the first layer that faces away from the second layer.

2. The method of claim 1, wherein foam forming the nonwoven web comprises depositing a first foam and a second foam onto a formation surface, the first foam including water and a plurality of first binder fibers, the second foam including water and a plurality of second binder fibers.

3. The method of claim 1, wherein the first layer comprises a plurality of first binder fibers, and the second layer comprises a plurality of second binder fibers and a plurality of superabsorbent particles.

4. The method of claim 1, wherein a water content in the second layer is greater than a water content in the first layer after the curing of the first layer.

5. The method of claim 1, wherein the first layer comprises a plurality of first binder fibers, the second layer comprises a plurality of second binder fibers, and the second binder fibers are different than the first binder fibers.

6. The method of claim 5, wherein the second binder fibers comprise PET fibers.

7. The method of claim 1, wherein the projections comprise a plurality of rounded ripples.

8. The method of claim 1, wherein a void volume of the first layer increases as the nonwoven web relaxes.

9. The method of claim 1, wherein the first layer is at least partially adhered to the second layer during the curing of the first layer.

10. The method of claim 1, wherein a thickness of the second layer is greater than a thickness of the first layer.

11. The method of claim 1, wherein:
at least partially curing the first layer comprises heating the first layer, a water content in the first layer decreasing during the heating of the first layer; and
curing the second layer comprises heating the second layer, a water content in the second layer decreasing during the heating of the second layer.

12. The method of claim 1, wherein at least partially curing the first layer comprises curing a first portion of the first layer at a rate different than a second portion of the first layer.

13. The method of claim 1, wherein the nonwoven web is a two-layer nonwoven web, and the plurality of layers consists of only the first and second layers.

14. A nonwoven article, comprising a nonwoven material manufactured according to the method of claim 1.

15. A nonwoven material, comprising:
a foam formed nonwoven web with a first layer and a second layer,
wherein the first layer is shrunken relative to the second layer such that the first layer forms a plurality of projections at an outer surface of the first layer that faces away from the second layer.

16. (canceled)

17. (canceled)

18. (canceled)

19. (canceled)

20. (canceled)

21. An absorbent article, comprising the nonwoven material of claim 15, wherein the outer surface of the first layer faces towards a wearer of the absorbent article when the absorbent article is worn by the wearer.

22. (canceled)

23. A method for forming a nonwoven material, comprising:
foam forming a nonwoven web with a plurality of layers, a first layer and a second layer of the plurality of layers at least partially interconnected; and
heating the nonwoven web such that the second layer contracts less than the first layer, the first layer deforming in order to produce a plurality of projections at an outer surface of the first layer that faces away from the second layer.

24. (canceled)

25. (canceled)

26. (canceled)

27. (canceled)

28. (canceled)

29. (canceled)

30. (canceled)

31. The method of claim 23, wherein the nonwoven web is a two-layer nonwoven web, and the plurality of layers consists of only the first and second layers.

32. A nonwoven article, comprising a nonwoven material manufactured according to the method of claim 23.

33. The method of claim 23, wherein the second layer comprises hydrogen-bond-capable fibers, and contraction of the second layer is at least partially due to capillary condensation during drying and hydrogen bonding.

34. The method of claim 33, wherein hydrogen-bond-capable fibers comprise wood pulp.

35. The method of claim 23, wherein the second layer comprises superabsorbent materials that are partially swollen after foam forming the nonwoven web, and contraction of the second layer is at least partially due to shrinkage of the superabsorbent materials during the heating the nonwoven web.

36. The method of claim 23, wherein the second layer comprises heat-shrinkable materials, and contraction of the second layer is at least partially due to shrinkage of the heat-shrinkable materials during the heating the nonwoven web.

37. The method of claim 36, wherein the heat-shrinkable materials comprises PLA staple fibers.
